(19) United States
(12) Patent Application Publication
(10) Pub. No.: US 2015/0265325 A1
(43) Pub. Date: Sep. 24, 2015

(54) Sternal Closure Apparatus, System and Method

(71) Applicant: Robert G Matheny, Norcross, GA (US)

(72) Inventor: Robert G Matheny, Norcross, GA (US)

(21) Appl. No.: 14/223,857

(22) Filed: Mar. 24, 2014

(51) Int. Cl. A61B 17/80; A61L 31/04; A61L 31/16

(52) U.S. Cl. CPC A61B 17/8076; A61L 31/16; A61L 31/041

(57) ABSTRACT

A bioresorbable closure member having integral first and second elongated projection members comprising first extracellular matrix (ECM) from a first mammalian tissue source, the first and second elongated projection members being configured wherein the longitudinal axes of the members intersect and form an angle with respect to said first and second members. The ECM can include one or more exogenously added biologically active agents, including osteogenic agents, bone morphogenic proteins, growth factors and cells.

### FIELD OF THE INVENTION

The present invention generally relates to surgical devices. More particularly, the present invention relates to apparatus, systems and methods for reapproximating the sternal halves of a patient's severed or separated sternum following a partial, medial or transverse sternotomy.

### BACKGROUND OF THE INVENTION

Various types of surgical procedures are currently performed to investigate, diagnose, and treat diseases involving a tissue or organs located in a patient's thoracic cavity (e.g., the heart, lungs, etc.). Using current techniques, many of the noted procedures typically require a partial or median sternotomy to gain access into the patient's thoracic cavity.

A partial or median sternotomy is a procedure by which a saw or other appropriate cutting instrument is used to make a midline incision along a portion or the entire axial length of the patient's sternum; allowing two opposing sternal halves to be separated laterally. A large opening into the thoracic cavity (or transversely to expose the mediastinal structures) is thus created, through which a surgeon may directly visualize and perform a surgical procedure on the heart and other thoracic organs or tissues.

Following the surgical procedure within the thoracic cavity, the two severed or separated sternal halves must be reapproximated (i.e. closed). Various conventional surgical methods have been employed to reapproximate the sternal halves.

One conventional surgical method, which is often employed, is to reapproximate the sternal halves with stainless steel wires that are wrapped around or through the sternal halves to exert medial compression thereon. The wires are also twisted together to approximate the sternum to secure the sternum.

In U.S. Pat. No. 5,423,821, selected plastics, nylon fibers and other expandable suturing materials are disclosed for closing the sternum. An associated suturing apparatus is also disclosed.

In U.S. Pat. No. 6,171,320, a surgical clip for closing the sternum is disclosed. The clip includes three lengths of a shape memory alloy wire; two of which form closed geometrical shapes similar in configuration and magnitude to each other, and the third connects the first two.

Further methods of sternum closure (and/or repair) include the use of band or strap assemblies. Such assemblies typically include a locking mechanism, which secures a band or strap in a closed looped configuration about desired sternum positions. Illustrative are the sternal closure assemblies disclosed in U.S. Pat. Nos. 5,417,698, 4,944,753, 5,356,412, 6,045,572 and 4,583,541.

U.S. Pat. No. 5,417,698 discloses a sternum closure assembly having two handles that are pivotally attached to one another and movable between an open and closed position. A retaining system configured to tighten wound closure material is also provided.

U.S. Pat. No. 4,944,753 discloses a method and device for producing an artificial retro-sternal tunnel or space, and an implantable elongate member for closure of the severed sternum.

U.S. Pat. No. 5,356,412 discloses a strap assembly that is configured to be looped about split portions of sternum. The strap assembly includes a flexible elongated member and a buckle member with a clamp element rotatably mounted within the buckle member from a non-strap securing position to a strap securing position in response to tensional forces exerted on the strap during tensioning thereof.

U.S. Pat. No. 6,045,572 discloses a sternum closure assembly that includes first and second grommets that are adapted to be disposed on two sides of the sternum, a wire suture, a mechanism for placing the grommets into the sternum and a placement tool.

Finally, U.S. Pat. No. 4,583,541 discloses a sternum closure device having an elongated strap-like member having a flat back surface that flatly overlies the anterior surface of a separated sternum. The forward surface of the member is convex and longitudinally grooved for nesting the tied end portions of suture wires across the posterior of the sternum.

While the use of steel wires and strap assemblies for sternum closure (and repair) have been widely accepted, there are a number of drawbacks and disadvantages associated therewith. A major disadvantage associated with steel wires is that the wires can, and often will, break.

Steel wires also provide insufficient and/or non-uniform clamping forces, resulting in sternal nonunion. Steel wires are also difficult to maneuver and place around the sternum.

Further, the small diameter of the steel wires can cause the wires to migrate into or through the tissue surrounding the sternum region or into the sternal bone itself over time. This can lead to significant patient pain and discomfort, in addition to slowing the postoperative recovery.

Major disadvantages associated with strap assemblies are (i) the strap mechanisms associated therewith are often structurally complex, and (ii) the strap assemblies are difficult to precisely position about the sternum.

There are also bone and surrounding tissue healing problems associated with the use of steel wires and band assemblies, due to improper forces exerted by these devices. The improper forces often cause undesirable bone movements, leading to raking and rubbing of surrounding tissue or bone.

A further surgical method that is often employed to effectuate sternum closure is to reapproximate the sternal halves with staples. Illustrative are the methods and associated stapling apparatus disclosed in U.S. Pat. Nos. 4,122,989, 5,163,598 and 6,030,410.

The sternum closure method disclosed in U.S. Pat. No. 6,030,410 comprises the steps of providing suture outlets in each sternal half and applying staples to the sternal halves to reinforce the bone adjacent to the suture outlets, whereby the sutures are prevented from cutting into the bone. As each suture loop is tightened, the suture loop abuts the staples adjacent the suture outlets.

There are similarly several drawbacks and disadvantages associated with the noted staple methods, and methods similar thereto. A major disadvantage is that the methods make reentry into the thoracic cavity through the sternotomy extremely difficult in the event of a medical emergency during or after a surgical procedure.

To overcome the problems inherent in direct fixation devices and methods, several alternative devices and methods of sternum closure have been proposed. Illustrative are the sternum closure devices and associated methods disclosed in U.S. Pat. Nos. 6,007,538 and 6,051,007.

U.S. Pat. No. 6,007,538 discloses overlapping sternum plates that can be removably joined to one another.

U.S. Pat. No. 6,051,007 discloses a separable sternum clamping apparatus that includes J or C-shaped sternum engagement legs. The sternum clamp plates are laterally adjustable relative to one another, but can be rigidly joined by, for example, a set of machine screws.

Although each of the noted sternum closure apparatus facilitates relatively easy reopening of the sternum, if necessary, the apparatus are similarly complex and difficult to use.

A further major problem that is associated with sternum closure apparatus and methods, including the apparatus and methods described above, is the risk of sternal infection. Sternal infection is a life-threatening complication, which is often encountered after cardiac surgery.

It would thus be desirable to provide sternum closure apparatus and associated methods for sternal reapproximation that provide a safe, reliable, stable and uniform clamping force and facilitate reopening of the sternum, if necessary, e.g., in the event of a medical emergency that requires re-entry into a patient's thoracic cavity.

It would further be desirable to provide sternum closure apparatus and associated methods for sternal reapproximation that substantially reduce the risk of infection after sternal reapproximation.

It would further be desirable to provide sternum closure apparatus and associated methods for sternal reapproximation that induce and/or active support healing or regeneration of bone and surrounding tissue.

It is therefore an object of the present invention to provide sternum closure apparatus and associated methods for sternal reapproximation that provide safe, reliable, stable and uniform clamping forces and facilitate reopening of the sternum, if necessary.

It is another object of the present invention to provide sternum closure apparatus and associated methods for sternal reapproximation that substantially reduce the risk of infection after sternal reapproximation.

It is another object of the present invention to provide sternum closure apparatus and associated methods for sternal reapproximation that induce or support healing or regeneration of bone and surrounding tissue.

It is another object of the present invention to provide sternum closure apparatus and associated methods for sternal reapproximation that also facilitates delivery of pharmacological agents to bone and surrounding tissues.

### SUMMARY OF THE INVENTION

The present invention provides improved apparatus and method for reapproximating the sternal halves of a patient's sternum following a median or partial sternotomy. The sternum closure apparatus facilitate ready access to the thoracic cavity during or after a medical procedure (e.g., in the event of a medical emergency) and overcome sternum nonunion problems inherent in conventional sternum closure apparatus.

In some embodiments of the invention, the sternum closure apparatus includes a plurality of elongated projecting (or projection) members.

In some embodiments, the elongated projecting members have linear longitudinal axes that intersect proximate the midpoint of each member.

According to the invention, the angle formed at the intersection of the longitudinal axes of the projecting members can be in the range of approximately 1°-45°.

In some embodiments of the invention, intersection of the longitudinal axes of the projecting members is approximately 45°, whereby the sternum closure apparatus comprises an X-shaped member.

In another embodiment, the sternum closure member comprises a multi-link member.

In some embodiments of the invention, the sternum closure members include at least one internal reservoir that is configured to receive and disperse a biologically active agent, i.e. an agent that induces or modulates a physiological or biological process, or cellular activity, e.g., induces growth and/or regeneration of sternum structures and tissue, or pharmacological agent, i.e. an agent or composition that is capable of producing a desired biological effect in vivo, e.g., stimulation or suppression of apoptosis, stimulation or suppression of an immune response, etc.

In a preferred embodiment of the invention, the sternum closure members comprise osteoinductive members.

In some embodiments of the invention, the sternum closure members comprise a biodegradable metal, such as magnesium.

In some embodiments, the sternum closure members comprise a shape memory material, such as Nitinol®.

In some embodiments, the sternum closure members comprise a degradable polymeric material, such as polylactic acid (PLA).

In some embodiments, the sternum closure members comprise a bone cement material, such as poly(methyl methacrylate) (PMMA).

In some embodiments of the invention, the metal, polymer and bone cement closure members are coated with a bioresorbable composition that includes a biologically active or pharmacological agent.

In some embodiments of the invention, the sternum closure members comprise an extracellular matrix (ECM) material. According to the invention, the ECM material can be derived from various mammalian tissue sources including, without limitation, small intestinal submucosa, stomach submucosa, large intestinal tissue, urinary bladder submucosa, urinary bladder membrane, liver basement membrane, cardiac tissue, e.g., pericardium, epicardium, endocardium and/or myocardium tissue, dura tissue, skin tissue, lung tissue, kidney tissue, pancreatic tissue, prostate tissue, mesothelial tissue, nervous system tissue, fetal tissue, placenta tissue, ureter tissue, cardiovascular tissue, e.g., veins and arteries, heart valves with or without their attached vessels, tissue surrounding the roots of developing teeth, and tissue surrounding growing bone.

In some embodiments, the ECM sternum closure members include one or more additional biologically active agents or compositions.

In some embodiments, the ECM sternum closure members include at least one pharmacological agent or composition.

According to the invention, the biologically active and pharmacological agents can be incorporated into the ECM material or coated on the ECM sternum closure members.

In some embodiments, the biologically active agent is selected from the group comprising proteins, growth factors and cells.

In some embodiments, the biologically active agent comprises an osteogenic agent.

In some embodiments, the pharmacological agent or composition is selected from the group comprising antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent specifically comprises an anti-microbial agent.

In some embodiments of the invention, the pharmacological agent comprises an anti-inflammatory agent.

In some embodiments of the invention, the pharmacological agent comprises a statin, i.e. a HMG-CoA reductase inhibitor.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of a sternum closure apparatus, in accordance with the invention;

FIG. 2 is perspective view of one embodiment of a sternum closure apparatus having an internal agent reservoir, in accordance with the invention;

FIG. 3 is perspective view of another embodiment of a sternum closure apparatus, in accordance with the invention; and

FIG. 4 is an illustration of a patient's sternum with the sternum closure apparatus shown in FIGS. 1 and 3 connected thereto, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### DEFINITIONS

The term “bioresorbable,” as used herein, means and includes a biocompatible material, composition or object that has the ability to be gradually integrated into a host. When used in the context of the sternum closure apparatus and systems of the invention, the term generally refers to the ability of at least a portion of a sternum closure apparatus to gradually be replaced by natural tissue, such replacement typically occurring naturally by the physiological process of remodeling.

The term “osteoanagenesis,” as used herein, means and includes the regeneration of bone tissue.

The term “osteoinductive,” as used herein, means and includes a structure, material or composition that induces and/or supports the formation, development and growth of new bone, and/or the remodeling of existing bone.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “biologically active agent”, as used herein, means and includes an agent that induces or modulates a physiological or biological process, or cellular activity, e.g., induces growth and/or regeneration of sternum structures and tissue. The term “biologically active agent” thus includes osteogenic agents selected from the group comprising, without limitation, polypeptide growth factors, such as, osteogenin, insulin-like growth factor (IGF)-1, TGF-β1, TGF-β2, TGF-β3, TGF-β4, TGF-β5, osteoinductive factor (OIF), basic fibroblast growth factor (bFGF), acidic fibroblast growth factor (aFGF), platelet-derived growth factor (PDGF), vascular endothelial growth factor (VEGF), growth hormone (GH), osteogenic protein-1 (OP-1).

The term “biologically active agent” also includes bone proteins selected from the group comprising, without limitation, alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin.

The term “biologically active agent” also includes bone morphogenic proteins selected from the group comprising, without limitation, BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15.

The term “biologically active agent” also includes cells selected from the group comprising, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stern cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The term “biologically active agent” also includes, without limitation, a platelet derived growth factor (PDGF), epidermal growth factor (EGF), transforming growth factor alpha (TGF-alpha), fibroblast growth factor-2 (FGF-2), hepatocyte growth factor (HGF), nerve growth factor (NGF), tumor necrosis factor alpha (TNA-alpha), and placental growth factor (PLGF).

The term “biologically active agent” also includes, without limitation, the following active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include anti-microbial agents, including, without limitation, anti-viral agents, anti-fungal agents and anti-parasites.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, Anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antiobiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “pharmacological agent”, “active agent”, “drug” and “active agent formulation” can further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and ifosfamide. Chemotherapy agents can also include, without limitation, antimetabolites, such as purine analogues, pyrimidine analogues and antifolates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab and trastuzumab.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifel one, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The terms “active agent formulation”, “pharmacological agent formulation” and “agent formulation”, are also used interchangeably herein, and mean and include an active agent optionally in combination with one or more pharmaceutically acceptable carriers and/or additional inert ingredients. According to the invention, the formulations can be either in solution or in suspension in the carrier.

The term “pharmacological composition”, as used herein, means and includes a composition comprising a “pharmacological agent” and/or a “pharmacological agent formulation” and/or a “biologically active agent” and/or any additional agent or composition identified herein.

The term “therapeutically effective”, as used herein, means that the amount of the “pharmacological composition” and/or “pharmacological agent” and/or “active agent formulation” and/or “biologically active agent” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a “pharmacological composition” or “pharmacological agent” or “active agent formulation” or “biologically active agent” to biological tissue.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As stated above, the present invention is directed to improved apparatus and method for reapproximating the sternal halves of a patient's sternum following a median, partial or transverse sternotomy. The sternum closure apparatus facilitate ready access to the thoracic cavity during or after a medical procedure and overcome sternal nonunion problems inherent in conventional sternum closure apparatus.

In a preferred embodiment of the invention, the sternum closure apparatus of the invention comprise osteoinductive members.

As indicated above, in one embodiment of the invention, the sternum closure apparatus include a plurality of elongated projecting members.

In some embodiments, the elongated projecting members have linear longitudinal axes that intersect proximate the midpoint of each member.

According to the invention, the angle formed at the intersection of the longitudinal axes of the projecting members can be in the range of approximately 1°-45°.

In some embodiments of the invention, intersection of the longitudinal axes of the projecting members is approximately 45°, whereby the sternum closure apparatus comprises an X-shaped member.

In some embodiments of the invention, the sternum closure apparatus include at least one internal reservoir that is configured to receive and disperse a biologically active agent, i.e. an agent that induces or modulates a physiological or biological process, or cellular activity, e.g., induces growth and/or regeneration of sternum structures and tissue, and/or pharmacological agent, i.e. an agent or composition that is capable of producing a desired biological effect in vivo, e.g., stimulation or suppression of apoptosis, stimulation or suppression of an immune response, etc.

In some embodiments of the invention, the noted sternum closure apparatus are coated with a bioresorbable composition that includes at least one biologically active or pharmacological agent.

According to the invention, the projecting members of the sternum closure apparatus can also have curved shapes.

In another embodiment, the sternum closure apparatus comprise a multi-link member, e.g., a plurality of rotatably connected projecting members.

In some embodiments of the invention, the sternum closure apparatus (and, hence, projecting members) comprise a biodegradable metal.

In some embodiments, the sternum closure apparatus (and, hence, projecting members) comprise a shape memory material.

In some embodiments, the sternum closure apparatus (and, hence, projecting members) comprise a degradable polymeric material.

In some embodiments, the sternum closure apparatus (and, hence, projecting members) comprise an osteoinductive composition comprising a base material or component and an ECM material.

According to the invention, the base material can comprise various natural and biocompatible materials, including, without limitation, autogenic bone, bone particulates, and calcium containing mineral compounds.

In some embodiments of the invention, the base material comprises bone marrow; preferably, bone marrow derived from heme producing bones. According to the invention, the bone marrow functions as a central supply for circulating cells; particularly, progenitor cells, which transition to stem cells.

In some embodiments of the invention, the base material comprises Artelon®, a degradeable polyurethane manufactured by Artimplant AB, Vastra Frolunda, Sweden.

In a preferred embodiment of the invention, the base material comprises autogenic bone, more preferably, particulate autogenic bone.

In some embodiments of the invention, the sternum closure apparatus (and, hence, projecting members) comprise an extracellular matrix (ECM) material (referred to hereinafter as “ECM sternum closure members”). According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety.

Suitable mammalian tissue sources include, without limitation, small intestinal submucosa, stomach submucosa, large intestinal tissue, urinary bladder submucosa, urinary bladder membrane, liver basement membrane, cardiac tissue, e.g., pericardium, epicardium, endocardium and/or myocardium tissue, dura tissue, skin tissue, lung tissue, kidney tissue, pancreatic tissue, prostate tissue, mesothelial tissue, nervous system tissue, fetal tissue, placenta tissue, ureter tissue, cardiovascular tissue, e.g., veins and arteries, heart valves with or without their attached vessels, tissue surrounding the roots of developing teeth, and tissue surrounding growing bone.

As indicated above, the ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

In some embodiments, the osteoinductive composition and/or ECM sternum closure members include one or more additional biologically active agents or compositions.

In some embodiments, the osteoinductive composition and/or ECM sternum closure members include at least one pharmacological agent or composition.

According to the invention, the biologically active and pharmacological agents can be incorporated into the ECM material or coated on the sternum closure members.

In some embodiments, the biologically active agent comprises one of the aforementioned biologically active agents, including, without limitation, a protein, growth factor and/or a cell.

In some embodiments, the biologically active agent comprises an osteogenic agent, i.e. an active agent that can elicit, facilitate and/or maintain the formation and growth of bone tissue. Suitable osteogenic agents include, without limitation, polypeptide growth factors, such as, osteogenin, Insulin-like Growth Factor (IGF)-1, TGF-β1, TGF-β2, TGF-β3, TGF-β4, TGF-β5, osteoinductive factor (OIF), basic Fibroblast Growth Factor (bFGF), acidic Fibroblast Growth Factor (aFGF), Platelet-Derived Growth Factor (PDGF), vascular endothelial growth factor (VEGF), Growth Hormone (GH), osteogenic protein-1 (OP-1) and any one of the many known bone morphogenic proteins (BMPs), including but not limited to BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15.

Suitable osteogenic agents further include, without limitation, extracellular matrix-associated bone proteins (e.g., alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin).

In some embodiments, the pharmacological agent or composition comprises one of the aforementioned pharmacological agents and compositions.

In some embodiments of the invention, the pharmacological agent specifically comprises an anti-microbial agent.

In some embodiments of the invention, the pharmacological agent comprises an anti-inflammatory agent.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

In some embodiments of the invention, the pharmacological agent comprises chitosan.

Referring now to FIG. 1, there is shown one embodiment of a sternum closure apparatus of the invention. As illustrated in FIG. 1, the closure apparatus 10 includes two integral linear projecting members 12, 14, having longitudinal axes L₁₂, L₁₄, respectively, that intersect proximate their midpoint to form angle A.

As indicated above, according to the invention, angle A can be in the range of 1°-45°. In the embodiment illustrated in FIG. 1, angle A is approximately 45°, whereby the apparatus 10 has an X shape.

The closure apparatus 10 further includes at least one, more preferably, a plurality of engagement lumens 16 that facilitate engagement of the sternum closure apparatus 10 to the sternum of a patient, e.g. via sutures.

According to the invention, the apparatus 10 can comprise various biocompatible materials, including the aforementioned materials and compositions, i.e. osteoinductive composition.

In some embodiments of the invention, the apparatus 10 comprises a biocompatible metal, including, without limitation, stainless steel.

In some embodiments, the apparatus 10 comprises a biodegradable metal. According to the invention, suitable biodegradable metals include, without limitation, magnesium and iron-based metals.

In some embodiments, the apparatus 10 comprises a shape memory material. According to the invention, suitable shape memory materials include, without limitation, Nitinol®, polyether ether ketone (PEEK) and poly(ethylene terephthalate) (PET).

In some embodiments, the apparatus 10 comprises a degradable polymeric material. According to the invention, suitable degradable polymeric materials include, without limitation, polylactic acid (PLA), poly-L-lactide (PLLA), and Artelon®.

In some embodiments of the invention, the apparatus 10 comprises an extracellular matrix (ECM) material. According to the invention, the ECM material can be derived from various mammalian tissue sources including, without limitation, small intestinal submucosa, stomach submucosa, large intestinal tissue, urinary bladder submucosa, urinary bladder membrane, liver basement membrane, cardiac tissue, e.g., pericardium, epicardium, endocardium and/or myocardium tissue, dura tissue, skin tissue, lung tissue, kidney tissue, pancreatic tissue, prostate tissue, mesothelial tissue, nervous system tissue, fetal tissue, placenta tissue, ureter tissue, cardiovascular tissue, e.g., veins and arteries, heart valves with or without their attached vessels, tissue surrounding the roots of developing teeth, and tissue surrounding growing bone.

In some embodiments, the apparatus 10 comprises an osteoinductive composition comprising a base material or component and an ECM material.

In some embodiments, the base material comprises autogenic bone.

In some embodiments, the base material comprises bone marrow; preferably, bone marrow derived from heme producing bones.

In some embodiments of the invention, wherein the apparatus 10 comprises an ECM material or an osteoinductive composition, the ECM includes a biologically active or pharmacological agent, e.g. impregnated therein.

In some embodiments of the invention, apparatus 10 is coated with a bioresorbable composition that includes at least one biologically active or pharmacological agent, such as an osteoinductive composition of the invention.

In some embodiments of the invention, wherein the apparatus 10 comprises a metal, osteoinductive composition or polymeric material, the apparatus 10 is coated with an ECM material.

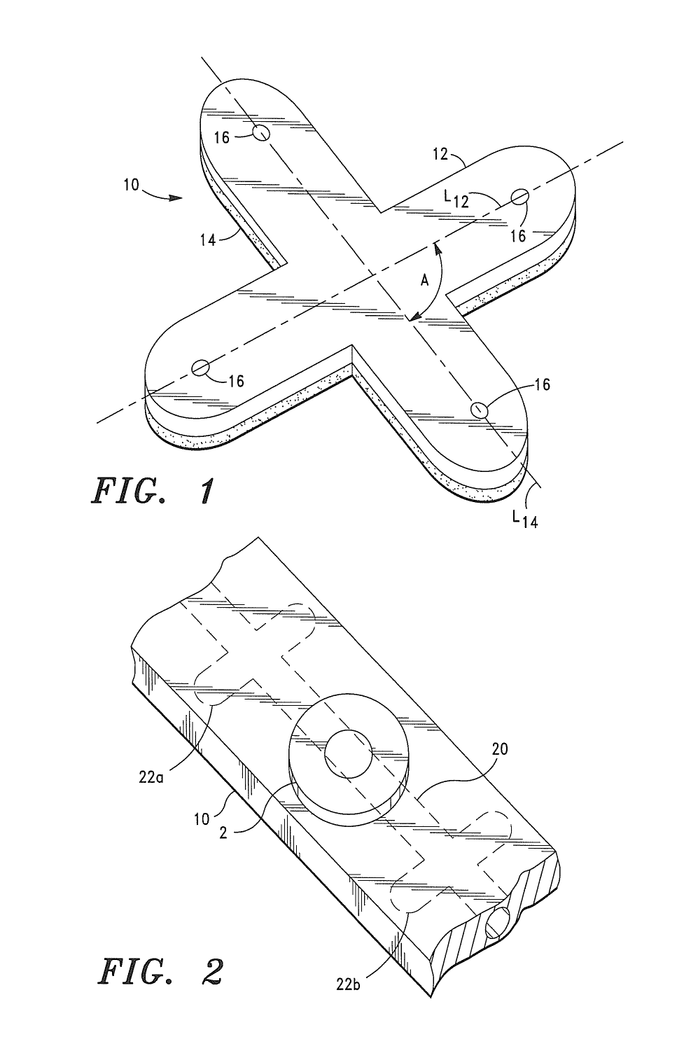

Referring now to FIG. 2, in some embodiments of the invention, the apparatus 10 (or an elongated projecting member) includes an internal reservoir 20 that is adapted to receive a biologically active or pharmacological agent therein. According to the invention, dispersal of the biologically active or pharmacological agent can be provided via one or more connecting lumens, e.g. lumens 22a, 22b.

According to the invention, the reservoir 20 can comprise a sealed structure, e.g., sealed with a material that degrades upon contact with bodily fluids. The reservoir 20 can also include a charging or inlet port 22 that facilitates on-going administration of biologically active or pharmacological agents to patient 100 when the sternum closure apparatus 10 is engaged to the patient's sternum 102 (see FIG. 4).

According to the invention, the biologically active agent referenced above can comprise any of the aforementioned biologically active agents.

In some embodiments, the biologically active agent comprises an osteogenic agent.

According to the invention, the pharmacological agent or composition can similarly comprise any of the aforementioned pharmacological agents and compositions, including antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents

In some embodiments of the invention, the pharmacological agent specifically comprises an anti-microbial agent.

In some embodiments of the invention, the pharmacological agent comprises an anti-inflammatory agent.

In some embodiments of the invention, the pharmacological agent comprises a statin, i.e. a HMG-CoA reductase inhibitor.

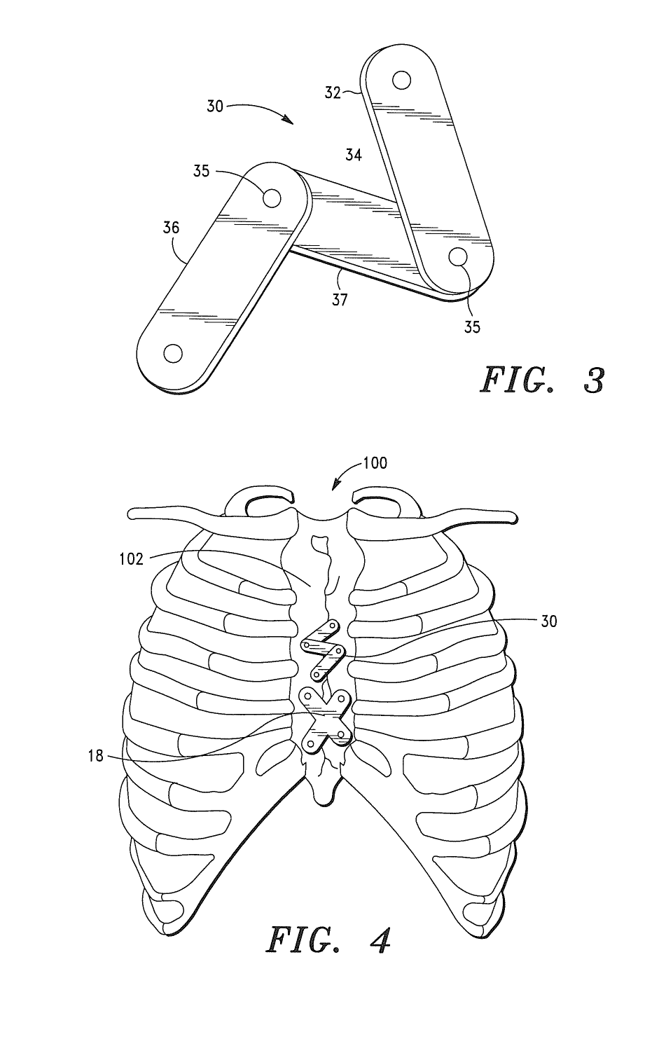

Referring now to FIG. 3, there is shown another embodiment of a sternum closure apparatus of the invention. As illustrated in FIG. 3, the apparatus 30 comprises a multi-link member having a plurality of rotatably connected or pivoting links 32, 34, 36. According to the invention, the pivoting points 35, 37 allow for flexibility in the location where the apparatus 30 is connected to a patient's sternum. The apparatus 30 thus provides the surgeon with flexibility at desired locations to employ one or more apparatus, e.g. apparatus 10 and/or 30 (see FIG. 4).

According to the invention, the links 32, 34, 36 can similarly comprise any of the aforementioned materials. The links 32, 34, 36 can also include internal reservoirs that are similarly configured to receive and disperse a biologically active or pharmacological agent and/or include biologically active and/or pharmacological agent and/or ECM material coatings.

Links 32, 34, 36 comprising an ECM material can also include a biologically active or pharmacological agent, e.g. impregnated therein.

As will readily be appreciated by one having ordinary skill in the art, the present invention provides numerous advantages compared to prior art prosthetic valves. Among the advantages are the following:
The provision of sternum closure apparatus and associated methods for sternal reapproximation that provide safe, reliable, staple and uniform clamping forces and facilitate reopening of the sternum, if necessary.
The provision of sternum closure apparatus and associated methods for sternal reapproximation that substantially reduce the risk of infection after sternal reapproximation.
The provision of sternum closure apparatus and associated methods for sternal reapproximation that induce or support healing or regeneration of bone and surrounding tissue.
The provision of sternum closure apparatus and associated methods for sternal reapproximation that also facilitates delivery of pharmacological agents to bone and surrounding tissues.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A sternal closure apparatus, comprising:
a bioresorbable closure member having integral first and second elongated projection members, said first elongated member having a first longitudinal axis, said second elongated projection member having a second longitudinal axis, said first and second elongated projection members being configured wherein said first and second longitudinal axes intersect and form a first intersecting angle with respect to said first and second elongated projection members,
said closure member comprising first extracellular matrix (ECM) from a first mammalian tissue source.

2. The sternal closure apparatus of claim 1, wherein said first mammalian tissue source is selected from the group consisting of small intestine submucosa (SIS), urinary bladder submucosa (UBS), stomach submucosa (SS), central nervous system tissue, mesodermal tissue, dermal extracellular matrix, subcutaneous extracellular matrix, gastrointestinal extracellular matrix, tissue surrounding growing bone, placental extracellular matrix, ornomentum extracellular matrix, cardiac extracellular matrix, kidney extracellular matrix, pancreas extracellular matrix, lung extracellular matrix, and combinations thereof.

3. The sternal closure apparatus of claim 2, wherein said first mammalian tissue source comprises an adolescent mammalian tissue source.

4. The sternal closure apparatus of claim 1, wherein said first ECM further comprises at least one exogenously added biologically active agent.

5. The sternal closure apparatus of claim 4, wherein said biologically active agent comprises an osteogenic agent selected from the group consisting of polypeptide growth factors, such as, osteogenin, insulin-like growth factor (IGF)-1, TGF-β1, TGF-β2, TGF-β3, TGF-β4, TGF-35, osteoinductive factor (OIF), basic fibroblast growth factor (bFGF), acidic fibroblast growth factor (aFGF), platelet-derived growth factor (PDGF), vascular endothelial growth factor (VEGF), growth hormone (GH), osteogenic protein-1 (OP-1).

6. The sternal closure apparatus of claim 4, wherein said biologically active agent comprises a bone protein selected from the group consisting of alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin.

7. The sternal closure apparatus of claim 4, wherein said biologically active agent comprises a bone morphogenic protein selected from the group consisting of BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15.

8. The sternal closure apparatus of claim 4, wherein said biologically active agent comprises a cell selected from the group consisting of a mesenchymal stem cell, embryonic stem cell, bone marrow cell, myofibroblast, skeletal myoblast, osteoblast, chondrocyte, and bone marrow-derived progenitor cell.

9. The sternal closure apparatus of claim 4, wherein said biologically active agent comprises a growth factor selected from the group consisting of a platelet derived growth factor (PDGF), epidermal growth factor (EGF), transforming growth factor alpha (TGF-alpha), fibroblast growth factor-2 (FGF-2), hepatocyte growth factor (HGF), nerve growth factor (NGF), tumor necrosis factor alpha (TNA-alpha), and placental growth factor (PLGF), and VEGF.

10. The sternal closure apparatus of claim 1, wherein said closure member comprises an osteoinductive composition comprising a base material comprising autogenic bone and a second ECM from a second mammalian tissue source selected from the group consisting of small intestine submucosa (SIS), urinary bladder submucosa (UBS), stomach submucosa (SS), central nervous system tissue, mesodermal tissue, dermal extracellular matrix, subcutaneous extracellular matrix, gastrointestinal extracellular matrix, tissue surrounding growing bone, placental extracellular matrix, ornomentum extracellular matrix, cardiac extracellular matrix, kidney extracellular matrix, pancreas extracellular matrix, lung extracellular matrix, and combinations thereof.

11. The sternal closure apparatus of claim 10, wherein said base material comprises bone marrow.

12. The sternal closure apparatus of claim 10, wherein said base material comprises Artelon®.

13. The sternal closure apparatus of claim 10, wherein said osteoinductive composition further comprises at least one exogenously added biologically active agent.

14. The sternal closure apparatus of claim 1, wherein said first intersecting angle is in the range of 1°-45°.

15. The sternal closure apparatus of claim 1, wherein said closure member includes at least one internal reservoir that is configured to receive and disperse a biologically active agent therefrom.

16. The sternal closure apparatus of claim 1, wherein said closure member comprises a biocompatible shape memory material.

17. The sternal closure apparatus of claim 14, wherein said biocompatible shape memory material comprises Nitinol®.

18. A sternal closure apparatus, comprising:
a multi-link bioresorbable closure member having a plurality of rotatably connected elongated projection members, said closure member comprising first extracellular matrix (ECM) from a first mammalian tissue source.

19. The sternal closure apparatus of claim 18, wherein said first mammalian tissue source is selected from the group consisting of small intestine submucosa (SIS), urinary bladder submucosa (UBS), stomach submucosa (SS), central nervous system tissue, mesodermal tissue, dermal extracellular matrix, subcutaneous extracellular matrix, gastrointestinal extracellular matrix, tissue surrounding growing bone, placental extracellular matrix, ornomentum extracellular matrix, cardiac extracellular matrix, kidney extracellular matrix, pancreas extracellular matrix, lung extracellular matrix, and combinations thereof.

20. The sternal closure apparatus of claim 19, wherein said first mammalian tissue source comprises an adolescent mammalian tissue source.

21. The sternal closure apparatus of claim 19, wherein said first ECM further comprises at least one exogenously added biologically active agent.

22. The sternal closure apparatus of claim 21, wherein said biologically active agent comprises an osteogenic agent selected from the group consisting of polypeptide growth factors, such as, osteogenin, insulin-like growth factor (IGF)-1, TGF-β1, TGF-β2, TGF-β3, TGF-β4, TGF-β5, osteoinductive factor (OIF), basic fibroblast growth factor (bFGF), acidic fibroblast growth factor (aFGF), platelet-derived growth factor (PDGF), vascular endothelial growth factor (VEGF), growth hormone (GH), osteogenic protein-1 (OP-1).

23. The sternal closure apparatus of claim 21, wherein said biologically active agent comprises a bone protein selected from the group consisting of alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin.

24. The sternal closure apparatus of claim 21, wherein said biologically active agent comprises a bone morphogenic protein selected from the group consisting of BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15.

25. The sternal closure apparatus of claim 18, wherein said closure member comprises an osteoinductive composition comprising a base material comprising autogenic bone and a second ECM from a second mammalian tissue source selected from the group consisting of small intestine submucosa (SIS), urinary bladder submucosa (UBS), stomach submucosa (SS), central nervous system tissue, mesodermal tissue, dermal extracellular matrix, subcutaneous extracellular matrix, gastrointestinal extracellular matrix, tissue surrounding growing bone, placental extracellular matrix, ornomentum extracellular matrix, cardiac extracellular matrix, kidney extracellular matrix, pancreas extracellular matrix, lung extracellular matrix, and combinations thereof.

26. The sternal closure apparatus of claim 25, wherein said osteoinductive composition further comprises at least one exogenously added biologically active agent.

27. The sternal closure apparatus of claim 18, wherein at least one of said plurality of elongated projection members includes at least one internal reservoir that is configured to receive and disperse a biologically active agent therefrom.
